(19) United States
(12) Patent Application Publication
(10) Pub. No.: US 2024/0254317 A1
(43) Pub. Date: Aug. 1, 2024

(54) DOPANT SOLUTION FOR CONDUCTIVE POLYMER, MONOMER LIQUID FOR PRODUCING CONDUCTIVE POLYMER, CONDUCTIVE COMPOSITION AND METHOD FOR PRODUCING SAME, AND ELECTROLYTIC CAPACITOR AND METHOD FOR PRODUCING SAME

(71) Applicant: TAYCA CORPORATION, Osaka-shi, Osaka (JP)

(72) Inventors: Yuhei Tsurumoto, Osaka-shi (JP); Megumi Seki, Osaka-shi (JP)

(73) Assignee: TAYCA CORPORATION, Osaka-shi, Osaka (JP)

(21) Appl. No.: 18/564,162

(22) PCT Filed: May 26, 2022
(86) PCT No.: PCT/JP2022/021491

§ 371 Date: Nov. 27, 2023

(30) Foreign Application Priority Data
May 31, 2021 (JP) 2021-091411

(51) Int. Cl. C08K 5/42; C07C 211/04; C07C 309/44; H01G 9/00; H01G 9/025

(52) U.S. Cl. CPC C08K 5/42; C07C 211/04; C07C 309/44; H01G 9/0036; H01G 9/025

(57) ABSTRACT

The present invention provides an electrolytic capacitor with excellent heat resistance, a method for manufacturing the same, a conductive composition that can constitute the electrolytic capacitor, a method for manufacturing the same, and a dopant solution and a monomer liquid for manufacturing the conductive composition. The dopant solution for a conductive polymer of the present invention comprises a dopant for a conductive polymer, the dopant dissolved in a solvent, and the dopant for the conductive polymer comprises a salt (A) of: a sulfonic acid having an anthraquinone skeleton; and one of specific alkylamine, specific alkanolamine, specific hydroxylamine and specific compound having a heterocycle having 1 to 3 nitrogen atoms in a ring thereof; and water or a lower alcohol is included as a solvent.

### TECHNICAL FIELD

The present invention relates to an electrolytic capacitor with excellent heat resistance, a method for manufacturing the same, a conductive composition that can constitute the electrolytic capacitor, a method for manufacturing the same, and a dopant solution and a monomer liquid for manufacturing the conductive composition.

### TECHNICAL BACKGROUND

Having its high conductivity, conductive polymers are used as electrolytes (solid electrolytes) in, for example, aluminum electrolytic capacitors, tantalum electrolytic capacitors, niobium electrolytic capacitors, and the like.

As the conductive polymer for this purpose, for example, those obtained by chemical oxidative polymerization or electrolytic oxidative polymerization of thiophene or its derivatives are used.

Organic sulfonic acids are mainly used as dopants in the chemical oxidative polymerization of thiophene or its derivatives, among which naphthalene sulfonic acids are often used. From the view point of improving the heat resistance of electrolytic capacitors, the application of sulfonic acids having an anthraquinone skeleton such as anthraquinone sulfonic acid is also being considered (See Patent Documents 1, 2, etc.).

### PRIOR ART REFERENCES

### Patent References

Patent Reference No. 1: Japanese Laid-Open Patent Publication No. 2000-12394

Patent Reference No. 2: Japanese Laid-Open Patent Publication No. 2007-142070

### SUMMARY OF INVENTION

### Objectives To Solve By The Invention

By the way, when manufacturing electrolytic capacitors, for example, monomers, oxidizing agents, dopants, etc. are attached to the capacitor element, and then the monomers are polymerized to form a conductive polymer (conductive composition) on the capacitor element. Although this is being used as a solid electrolyte, sulfonic acid having an anthraquinone skeleton has a high acidity, and depending on the material, the problem of corrosion of capacitor elements may occur.

On the other hand, as shown in Patent Document 1, if a salt such as sodium anthraquinone sulfonate or ammonium anthraquinone sulfonate is used, the problem of corrosion of the capacitor element as described above can be avoided; These salts have extremely low solubility in lower alcohols, which are commonly used as solvents for polymerization of conductive polymers, and in water, which is used as a solvent when forming dopants into solutions. Therefore, when using salts such as sodium anthraquinone sulfonate or ammonium anthraquinone sulfonate as a dopant, the amount of dopant that can be introduced into a conductive polymer in one polymerization is limited, so it is difficult to use a conductive polymer with excellent conductivity. The amount of polymer that can be formed in one polymerization is also limited, and in order to form a solid electrolyte layer of an electrolytic capacitor, it is necessary to repeat the polymerization many times.

Therefore, there is a need to develop a technology that improves the heat resistance of electrolytic capacitors while avoiding the above problems.

The present invention has been made in view of the above circumstances, and its objects are to provide an electrolytic capacitor with excellent heat resistance, a method for manufacturing the same, a conductive composition that can constitute the electrolytic capacitor and a method for manufacturing the same, and a dopant solution and a monomer solution for producing the conductive composition described above.

### Means to Solve the Objectives

The dopant solution for conductive polymers of the present invention (hereinafter sometimes simply referred to as “dopant solution”) has dissolved the dopant for conductive polymers in a solvent; and it contains a salt (A) of: a sulfonic acid having an anthraquinone skeleton; and one selected from the group consisting of an alkylamine represented by the following general formula (1), an alkanolamine represented by the following general formula (2), a hydroxylamine represented by the following general formula (3), and a compound having a heterocycle having 1 to 3 nitrogen atoms in a ring thereof. As the solvent therefor, water or a lower alcohol is included

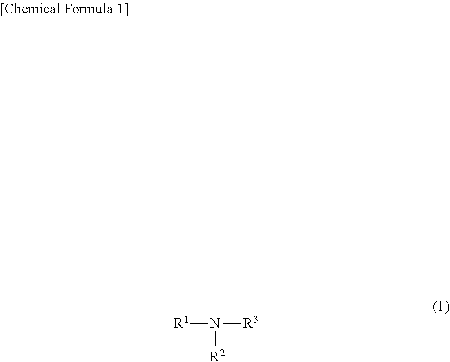

In the above general formula (1), R¹ and R² are each an alkyl group having 1 to 6 carbon atoms, and R³ is a hydrogen atom or an alkyl group having 1 to 6 carbon atoms.

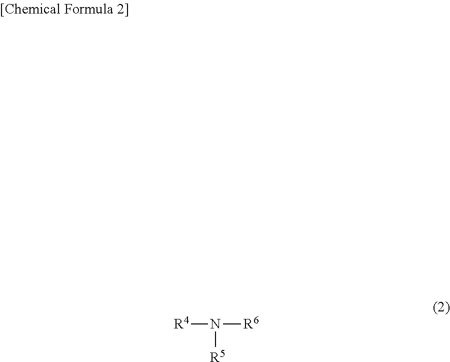

In the above general formula (2), R⁴ is a hydroxyalkyl group having 1 to 6 carbon atoms, and each of R⁵ and R⁶ is a hydrogen atom, a hydroxyalkyl group having 1 to 6 carbon atoms, or an alkyl group having 1 to 6 carbon atoms.

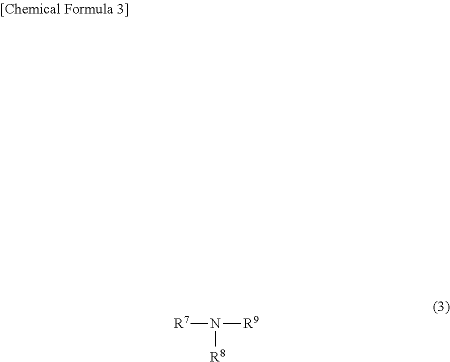

In the above general formula (3), R⁷ is a hydroxyl group, and each of R⁸ and R⁹ is an alkyl group having 1 to 6 carbon atoms.

Also, the monomer liquid for producing the conductive polymer of the present invention (hereinafter sometimes simply referred to as “monomer liquid”) contains a monomer for producing the conductive polymer and a dopant for the conductive polymer. The dopant for conductive polymer is dissolved. As the dopant for the conductive polymers, it contains a salt (A) of: a sulfonic acid having an anthraquinone skeleton; and one selected from the group consisting of an alkylamine represented by the above general formula (1), an alkanolamine represented by the above general formula (2), a hydroxylamine represented by the above general formula (3), and a compound having a heterocycle having 1 to 3 nitrogen atoms in a ring thereof.

Also, the conductive composition of the present invention is obtained by oxidation polymerization of the monomer for producing the conductive polymer in the presence of a dopant solution for the conductive polymer of the present invention, or it is obtained by oxidation polymerization of the monomer for producing the conductive polymer by using the monomer liquid for producing the conductive polymer of the present invention.

Also, the method for producing the conductive composition of the present invention is characterized in oxidation polymerization of the monomer for producing the conductive polymer in the presence of the dopant solution for the conductive polymer of the present invention, or it is characterized in oxidation polymerization of the monomer for producing the conductive polymer by using the monomer liquid for producing the conductive polymer of the present invention.

Also, the electrolytic capacitor of the present invention is characterized by having the conductive composition of the present invention as a solid electrolyte.

The method of manufacturing an electrolytic capacitor of the present invention is characterized in that the conductive composition produced by the manufacturing method of the present invention is used as a solid electrolyte.

### Effects of the Invention

The present invention can provide an electrolytic capacitor with excellent heat resistance, a method for manufacturing the same, a conductive composition that can constitute the electrolytic capacitor, a method for manufacturing the same, and a dopant solution and a monomer liquid for manufacturing the conductive composition.

### Embodiments To Carry Out The Invention

### <Dopant Solution For Conductive Polymer>

The dopant solution of the present invention has dissolved the dopant for conductive polymers in a solvent; and it contains a salt (A) of: a sulfonic acid having an anthraquinone skeleton; and one selected from the group consisting of an alkylamine represented by the above general formula (1), an alkanolamine represented by the above general formula (2), a hydroxylamine represented by the above general formula (3), and a compound having a heterocycle having 1 to 3 nitrogen atoms in a ring thereof; and as the solvent therefor, water or a lower alcohol is included. By carrying out oxidation polymerization of the monomer for producing the conductive polymer using the dopant solution, a conductive composition containing a conductive polymer and a component derived from the above salt (A) can be obtained.

Unlike sodium anthraquinone sulfonate, ammonium anthraquinone sulfonate, etc., the above salt (A) has a high solubility in water or a lower alcohol. Therefore a dopant solution containing the above salt (A) at a high concentration of, for example, 5% by mass or more can be prepared, and an amount of a conductive composition with excellent conductivity [a conductive polymer containing a dopant derived from the above salt (A), etc.] that can be produced in one polymerization, can be increased. Furthermore, unlike anthraquinone sulfonic acid, it is unlikely to cause corrosion even when applied to a capacitor element made of a material such as aluminum that is easily corroded by the action of acids.

By using the conductive composition containing the dopant derived from the above salt (A) as a solid electrolyte, an electrolytic capacitor with excellent heat resistance can be obtained.

The above salt (A) used in the dopant solution is formed of a sulfonic acid having an anthraquinone skeleton; and one selected from the group consisting of an alkylamine represented by the above general formula (1), an alkanolamine represented by the above general formula (2), a hydroxylamine represented by the above general formula (3), and a compound having a heterocycle having 1 to 3 nitrogen atoms in a ring thereof.

Examples of the sulfonic acid having an anthraquinone skeleton forming the above salt (A) include anthraquinone sulfonic acids such as anthraquinone-1-sulfonic acid and anthraquinone-2-sulfonic acid; and anthraquinone disulfonic acids such as anthraquinone-1,5-disulfonic acid, anthraquinone-1,8-disulfonic acid, anthraquinone-2,6-disulfonic acid, and anthraquinone-2,7-disulfonic acid.

Examples of the alkylamine represented by the above general formula (1) forming the above salt (A) include secondary alkylamines such as dimethylamine, diethylamine, dipropylamine, dibutylamine, dipentylamine, and dihexylamine; and tertiary alkylamines such as trimethylamine, triethylamine, tripropylamine, tributylamine, tripentylamine, and trihexylamine.

Examples of the alkanolamine represented by the above general formula (2) that forms the above salt (A) include primary alkanolamines such as methanolamine, ethanolamine, propanolamine, butanolamine, pentanolamine, hexanolamine, 1,2-propanediolamine, and etc.; secondary alkanolamines such as dimethanolamine, diethanolamine, dipropanolamine, dibutanolamine, dipentanolamine, dihexanolamine, butanolethanolamine and etc.; and tertiary alkanolamines such as trimethanolamine, triethanolamine, tripropanolamine, tributanolamine, tripentanolamine, trihexanolamine, butyldihydroxylethylamine, dimethylhydroxylethylamine and etc.

Examples of the hydroxylamine represented by the above general formula (3) that forms the above salt (A) include diethylhydroxylamine.

Examples of the compound having a heterocycle containing 1 to 3 nitrogen atoms in the ring which forms the above salt (A) include imidazoles such as imidazole, 1-methylimidazole, 2-methylimidazole, 2-ethylimidazole, 2-butyl Imidazole, 2-undecylimidazole, 2-phenylimidazole, 4-methylimidazole, 4-undecylimidazole, 4-phenylimidazole, 2-ethyl-4-methylimidazole, 1,2-dimethylimidazole, and etc.

The above salt (A) above is obtained, for example, by: dissolving a sulfonic acid having an anthraquinone skeleton dissolved in water, followed by neutralizing with a neutralizing agent the alkylamine represented by the above general formula (1), the alkanolamine represented by the above general formula (2), the above-mentioned hydroxylamine represented by the above general formula (3), or the compound having the heterocycle containing 1 to 3 nitrogen atoms in the ring; or by reacting it with a salt of the alkanolamine represented by the above general formula (2) (e.g., a phosphate).

Since the alkylamine represented by the above general formula (1), the alkanolamine represented by the above general formula (2), the hydroxylamine represented by the above general formula (3), and the compound having the heterocycle containing 1 to 3 nitrogen atoms in the ring, which is used as a neutralizing agent to form the above salt (A), makes it possible to obtain a salt (A) having a higher solubility in water, it is preferable that the base dissociation constant pKb thereof is 6 or more, and 12 or less.

The dopant solution can contain only one kind of the above salts (A), or can contain two or more kinds.

Water or a lower alcohol is used as a solvent for the dopant solution. Also, examples of the lower alcohols include alcohols having 1 to 4 carbon atoms such as methanol, ethanol, propanol, and butanol. In the dopant solution, only one kind of the above-mentioned various solvents can be used, or two or more kinds can be used.

The concentration of the above salt (A) in the dopant solution is preferably 5% by mass or more, more preferably 10% by mass or more, and yet more preferably 20% by mass or more, from the viewpoint of increasing the polymerization efficiency of the conductive polymer with excellent conductivity. Also, there is no particular restriction on the upper limit of the concentration of the above salt (A) in the dopant solution, but it is usually about 50% by mass.

The dopant solution can also contain components other than the above salt (A) and the solvent. Examples of such components include an emulsifier. By containing an emulsifier in the dopant solution, the polymerization reaction of the conductive polymer can proceed more uniformly.

Although various emulsifiers can be used as the emulsifier, alkylamine oxides are particularly preferred. The alkylamine oxides, even if it remains in the conductive composition, do not greatly reduce the conductivity of the conductive composition, nor significantly deteriorate the functionality of an electrolytic capacitor when the conductive composition is used as a solid electrolyte of the electrolytic capacitor. The alkyl group in the alkylamine oxides described above preferably has 1 to 20 carbon atoms. Also, as the polymerization reaction of thiophene or its derivatives progresses, the pH of the reaction system decreases, but alkylamine oxides have an effect of suppressing the decrease of the pH. Therefore, it is particularly effective to use the above-mentioned alkylamine oxides in a dopant solution when the base material used to form the conductive polymer (the base material on which the above-mentioned precipitate is deposited, the capacitor element, etc.) does not have very good acid resistance.

The concentration of the emulsifier in the dopant solution is preferably 0.01 to 2% by mass, for example.

### <Monomer Liquid For Conductive Polymer Production>

The monomer liquid for producing a conductive polymer of the present invention contains a monomer for producing a conductive polymer (hereinafter sometimes simply referred to as “monomer”) and a dopant for the conductive polymer, and it has dissolved the dopant for the conductive polymer and contains the above salt (A) as the dopant for the conductive polymer.

Thiophene or its derivatives, pyrrole or its derivatives, and aniline or its derivatives, which are commonly used as monomers for producing conductive polymers, are liquid at room temperature, but the above salt (A) has a good solubility not only in water and lower alcohols used as a solvent for the dopant solution, but also in these monomers. Therefore, even when the monomer liquid is composed of only the monomer for producing a conductive polymer and the above salt (A), the monomer liquid can be made it have a high concentration of the above salt (A).

Also, when a lower alcohol is used as a solvent in the monomer liquid, or when it is used in the monomer liquid in the form of the dopant solution of the present invention containing the above salt (A), the concentration of the above salt (A) in the monomer liquid can be made higher.

Therefore, with the monomer liquid of the present invention, a conductive polymer with excellent conductivity (a conductive composition containing a conductive polymer and a dopant, etc.) can be efficiently produced, and by using this conductive polymer (conductive composition) as a solid electrolyte, an electrolytic capacitor with excellent heat resistance can be formed.

Examples of monomers used in the monomer liquid include thiophene or its derivatives, pyrrole or its derivatives, aniline or its derivatives, and one or more of these can be used, but in particular thiophene or its derivatives can be preferably used. This is because the conductive polymer obtained by polymerizing thiophene or its derivatives has a good balance of conductivity and heat resistance, making it easier to obtain electrolytic capacitors with superior capacitor characteristics compared to other monomers.

Examples of thiophene or thiophene derivatives include 3,4-ethylenedioxythiophene (EDOT), 3-alkylthiophene, 3-alkoxythiophene, 3-alkyl-4-alkoxythiophene, 3,4-alkylthiophene, 3,4-alkoxythiophene, and alkylated ethylenedioxythiophene (alkylated EDOT) obtained by modifying the above-mentioned 3,4-ethylenedioxythiophene with an alkyl group, in which the carbon number of the alkyl group and alkoxy group has a carbon number of preferably 1 or more, and preferably 16 or less, more preferably 10 or less, and yet more preferably 4 or less.

To explain in detail the alkylated EDOT obtained by modifying the above EDOT with an alkyl group, EDOT and alkylated EDOT correspond to the compound represented by the following general formula (4).

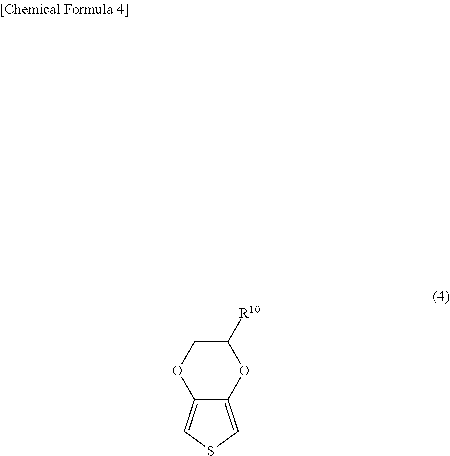

In the general formula (4), R¹⁰ is hydrogen or an alkyl group having 1 to 10 carbon atoms.

The compound when R¹⁰ in the above general formula (4) is hydrogen is EDOT, and when expressed by an IUPAC name, it is “2,3-dihydro-thieno[3,4-b][1,4]dioxin (2,3-Dihydro-thieno[3,4-b][1,4]dioxine)”, but this compound is more commonly known as “3,4-ethylenedioxythiophene” than its IUPAC name. In this specification, this “2,3-dihydro-thieno[3,4-b][1,4]dioxin” is expressed as “3,4-ethylenedioxythiophene (EDOT).” When R¹⁰ in the above general formula (4) is an alkyl group, the alkyl group preferably has 1 to 10 carbon atoms, particularly preferably 1 to 4 carbon atoms. That is, as the alkyl group, methyl group, ethyl group, propyl group, and butyl group are particularly preferable. To give specific examples of these, a compound when R¹⁰ in general formula (4) is a methyl group is “2-Methyl-2,3-dihydro-thieno[3,4-b][1,4]dioxin (2-Methyl-2,3-dihydro-thieno[3,4-b][1,4]dioxine) when it is expressed by the TUPAC name, but hereinafter this will be simply referred to as “methylated ethylenedioxythiophene (methylated EDOT).” The compound when R¹⁰ in the general formula (4) is an ethyl group is “2-ethyl-2,3-dihydro-thieno[3,4-b][1,4]dioxin (2-Ethyl-2,3-dihydro-thieno[3,4-b][1,4]dioxine)” when it is expressed by the IUPAC name, but in this specification, it is simplified referred to as “ethylated ethylenedioxythiophene (ethylated EDOT).”

The compound when R¹⁰ in the general formula (4) is propyl group is “2-propyl-2,3-dihydro-thieno[3,4-b][1,4]dioxin (2-propyl-2,3-dihydro-thieno[3,4-b][1,4]dioxine)” when it is expressed by the IUPAC name, but in this specification, it is simplified referred to as “propylated ethylenedioxythiophene (propylated EDOT).” Also, the compound when R¹⁰ in general formula (4) is butyl group is “2-butyl-2,3-dihydro-thieno[3,4-b][1,4]dioxin (2-Butyl-2,3-dihydro-thieno[3,4-b][1,4]dioxine)” when it is expressed by the IUPAC name, but in this specification, it is simplified referred to as “butylated ethylenedioxythiophene (butyl EDOT).” Also, in this specification, “2-alkyl-2,3-dihydro-thieno[3,4-b][1,4]dioxin” is simply referred to as “alkylated ethylenedioxythiophene (alkylated EDOT).” Among these alkylated EDOTs, methylated EDOT, ethylated EDOT, propylated EDOT, and butylated EDOT are preferred.

Also, EDOT (i.e., 2,3-dihydro-thieno[3,4-b][1,4]dioxin) and an alkylated EDOT (i.e., 2-alkyl-2,3-dihydro-thieno[3,4-b) [1,4]dioxin) is preferably used in combination, and the mixing ratio thereof is preferably 0.05:1 to 1:0.1 in terms of molar ratio, and more preferably 0.1:1 to 1:0.1, and yet more preferably 0.2:1 to 1:0.2, and particularly preferably 0.3:1 to 1:0.3.

Since monomers such as thiophene or its derivatives, pyrrole or its derivatives, and aniline or its derivatives are liquid at room temperature, it is possible to prepare a monomer liquid only using these monomers and the above salt (A), but it is possible to prepare a monomer liquid further including a solvent in order to smoothly proceed the polymerization reaction.

As the solvent for the monomer liquid, lower alcohols (alcohols having 1 to 4 carbon atoms such as methanol, ethanol, propanol, and butanol) are preferred.

In the monomer liquid, the ratio of the above salt (A) to the monomer is preferably 5:1 to 15:1 on a mass basis.

The concentration of the above salt (A) in the monomer liquid is preferably 5% by mass or more, more preferably 10% by mass or more, and yet more preferably 20% by mass or more, from the viewpoint of increasing the polymerization efficiency of the conductive polymer with excellent conductivity. Also, there is no particular restriction on the upper limit of the concentration of the above salt (A) in the monomer liquid, but it is usually about 50% by mass.

Further, when a solvent is used in the monomer liquid, the concentration of the monomer is usually 15 to 50% by mass.

The monomer liquid is prepared, for example, by mixing the monomer and the above salt (A) to dissolve the above salt (A) in the monomer; by mixing the monomer, the above salt (A) and a solvent to dissolve the monomer and the salt in the solvent; by mixing a monomer into the dopant solution of the present invention.

### <Conductive Composition>

The conductive composition of the present invention can be obtained by oxidation polymerization of a monomer for producing a conductive polymer in the presence of the dopant solution of the present invention, or by oxidation polymerization of a monomer for producing a conductive polymer using the monomer liquid of the present invention. The conductive composition obtained thereby contains a conductive polymer formed by polymerizing the monomers, and a component derived from the above salt (A) serving as a dopant.

More specifically, the conductive composition can be obtained, for example, by the following method (a) or (b).

### Method (a):

Step (a-1): First, a dopant solution of the present invention is applied to a base material such as a capacitor element on which a conductive composition is to be formed.

In addition to the capacitor element, a ceramic plate or the like can be used as a base material when a film made of a conductive composition is to be obtained.

There are no particular limitations on the method of applying the dopant solution to the base material, and therefore a method of immersing the base material in the dopant solution, a method of applying the dopant solution to the base material by spray coating, etc. can be adopted.

Also, after applying the dopant solution to the base material, the solvent of the dopant solution can be removed by drying as necessary.

Step (a-2): The monomer is attached to the base material that has undergone step (a-1).

There are no particular restrictions on the method of attaching the monomer to the base material. For example, a method of immersing the base material in the liquid monomer or the diluted solution in which the monomer is diluted with a solvent (the same solvent as the monomer liquid of the present invention can be used) and pulling it up, or a method of coating the base material by spraying the liquid monomer or the above-explained diluted solution, etc. can be adopted.

It is preferable that the amount of the monomer to be attached to the base material is at the ratio of the above salt (A): the monomer being 5:1 to 15:1 on a mass basis.

Also, after the monomer is attached to the base material, the solvent in the monomer liquid and the solvent in the dopant solution can be removed by drying as necessary.

Step (a-3): An oxidizing agent is attached to the base material to which the above salt (A) and the monomer have been attached through step (a-2), and then an oxidation polymerization is performed to form a conductive composition on the base material form things.

Examples of the oxidizing agents that can be used include persulfates such as ammonium persulfate, sodium persulfate, potassium persulfate, calcium persulfate, and barium persulfate; iron-based oxidizing agents such as ferric sulfate, ferric chloride, and ferric nitrate, and etc.

Per 1 mol of the above salt (A) as the dopant, the amount of the oxidizing agent to be used is, for example, preferably 0.4 mol or more, more preferably 0.5 mol or more, and preferably 4.0 mol or less, more preferably 3.5 mol or less.

There are no particular restrictions on the method of attaching the oxidizing agent to the base material. For example, a method of preparing a solution (for example, an aqueous solution) in which an oxidizing agent is dissolved, into which a base material is immersed, and then the base material is pulled up and dried, or a method in which the oxidizing agent solution is applied to the base material by spray coating or the like and then dried, etc. can be adopted.

The oxidation polymerization can be carried out, for example, at 5 to 95° C. for 1 to 72 hours.

After the oxidation polymerization is completed, the base material on which the conductive composition is formed is washed and dried.

When producing a conductive composition by the method (a), the above steps (a-1) to (a-3) can be repeated multiple times as necessary. For example, when forming a layer of the conductive composition on the surface of the capacitor element to function as a solid electrolyte of an electrolytic capacitor, the above steps (a-1) to (a-3) can be repeated multiple times, thereby making it possible to form a solid electrolyte layer with better properties.

It is noted that when a salt such as sodium anthraquinone sulfonate or ammonium anthraquinone sulfonate is used as a dopant, its solubility in the dopant solution is low and it is difficult to increase its concentration, and therefore in order to manufacture a conductive composition with high conductivity to form a solid electrolyte layer of an electrolytic capacitor, it is necessary to repeat the polymerization many times. However, in the case of the dopant solution of the present invention, the concentration of the above salt (A) can be adjusted to a high concentration as described above, and therefore even if the number of polymerizations [the number of repetitions from step (a-1) to step (a-3) in the case of the method (a)] is reduced, a solid electrolyte layer with high conductivity can be formed efficiently.

### Method (b):

Step (b-1): First, the monomer liquid of the present invention is applied to a base material such as a capacitor element (which is similar to the base material that can be used in method (a)) on which a conductive composition is to be formed.

There are no particular limitations on the method of applying the monomer liquid to the base material. For example, a method of immersing the base material in the monomer liquid or a method of applying the monomer liquid to the base material by spray coating or the like can be adopted.

Step (b-2): An oxidizing agent is attached to the base material that has undergone step (b-1).

The specific example of the oxidizing agent and its usage amount are the same as in the case of method (a). Also, as a method for attaching the oxidizing agent to the base material, the same method as described in step (a-3) can be adopted.

Step (b-3): The monomer attached to the base material after step (b-2) is undergone is polymerized by oxidation polymerization to form a conductive composition on the base material.

The conditions of the oxidation polymerization can be the same as in step (a-3). Also, after the oxidation polymerization is completed, the base material on which the conductive composition is formed is washed and dried.

When a conductive composition is produced by method (b), the above steps (b-1) to (b-3) can be repeated multiple times as necessary. For example, when forming a layer of a conductive composition on the surface of a capacitor element to function as a solid electrolyte of an electrolytic capacitor, the above steps (b-1) to (b-3) cab be repeated multiple times, thereby making it possible to form a solid electrolyte layer with better properties.

In addition, since the concentration of the above salt (A) as a dopant can be increased by using the monomer liquid of the present invention as described above, even if the number of repetition of the polymerization [number of repetition of the polymerization from step (b-1) to step (b-3) in the case of method (b)] is reduced, a layer of solid electrolyte having high conductivity can be efficiently formed, in the same manner as method (a).

### <Electrolytic Capacitor>

The electrolytic capacitor of the present invention has the conductive composition of the present invention as a solid electrolyte.

Examples of the electrolytic capacitor of the present invention include aluminum electrolytic capacitors such as wound type aluminum electrolytic capacitors, laminated type or flat plate type aluminum electrolytic capacitor; tantalum electrolytic capacitors; niobium electrolytic capacitors; and the like.

For example, in the case of a wound aluminum electrolytic capacitor, it is preferable that the capacitor element is made by: etching the surface of an aluminum foil; a lead terminal is attached to the anode on which a dielectric layer has been formed by chemical conversion treatment; another lead terminal is attached to the cathode made of aluminum foil; and the anode and cathode with the lead terminals are wound with a separator interposed therebetween.

Further, a wound aluminum electrolytic capacitor using the above capacitor element is manufactured, for example, as follows.

A solid electrolyte layer made of a conductive composition is formed on the surface of the above capacitor element by, for example, the method (a) or method (b) described above. Then, the capacitor element with the solid electrolyte layer formed thereon is then packaged with an exterior material to produce a wound aluminum electrolytic capacitor.

When electrolytic capacitors other than the above-mentioned wound type aluminum electrolytic capacitors, such as multilayer or flat plate aluminum electrolytic capacitors, tantalum electrolytic capacitors, and niobium electrolytic capacitors, are manufactured, a capacitor element having an anode made of a porous valve metal such as aluminum, tantalum, and niobium, and a dielectric layer made of an oxide film of the valve metal is used. In the same manner as the above-mentioned wound type aluminum electrolytic capacitors, the capacitor element is subject to the above-mentioned method (a) or method (b) to form a solid electrolyte layer made of a conductive composition. Then, carbon paste or silver paste is applied to the capacitor element on which the solid electrolyte layer has been formed, followed by drying it and packaging it to produce a laminated or flat plate aluminum electrolytic capacitor, tantalum electrolytic capacitor, niobium electrolytic capacitor, etc.

In addition, in manufacturing electrolytic capacitors, after manufacturing a conductive composition on a base material as described above, it is also possible to form a conductive polymer layer by applying a dispersion liquid of a x-conjugated conductive polymer, thereby making an electrolytic capacitor having the both serving as a solid electrolyte.

As the above-mentioned x-conjugated conductive polymer, a x-conjugated conductive polymer using a polymer anion as a dopant is used. This polymer anion is mainly composed of polymeric sulfonic acids. For example, specific examples thereof include polystyrene sulfonic acid, sulfonated polyester, phenolsulfonic acid novolak resin, and copolymer of styrene sulfonic acid and a non-sulfonic acid monomer (methacrylic acid ester, acrylic acid ester, and unsaturated hydrocarbon-containing alkoxysilane compounds or hydrolysates thereof, etc.).

In addition, it is possible that the solid electrolyte of the electrolytic capacitor contains: a high-boiling organic solvent with a boiling point of 150° C. or higher; or a conductive auxiliary liquid including a combination of a high-boiling organic solvent with a boiling point of 150° ° C. or higher, and an aromatic compound having at least one hydroxyl group or carboxyl group.

Examples of high-boiling organic solvents with a boiling point of 150° C. or higher that can be used in the conductive auxiliary liquid include γ-butyrolactone (boiling point: 203° C.), butanediol (boiling point: 230° C.), dimethyl sulfoxide (boiling point: 189ºC), sulfolane (boiling point: 285° C.). N-methylpyrrolidone (boiling point: 202° C.), dimethylsulfolane (boiling point: 233° C.), ethylene glycol (boiling point: 198° C.), diethylene glycol (boiling point: 244° C.), triethyl phosphate (boiling point: 215° C.), tributyl phosphate (289° C.), triethylhexyl phosphate [215° C. (4 mmHg)], polyethylene glycol, and the like.

Also, as the above-mentioned aromatic compounds having at least one of hydroxyl group (which corresponds to a hydroxyl group bonded to a constituent carbon of an aromatic ring, and does not mean an —OH moiety such as in a carboxyl group) and a carboxyl group, ones of a benzene-based, a naphthalene-based, and an anthracene-based can be used. Specific examples thereof include hydroxybenzenecarboxylic acid, nitrophenol, dinitrophenol, trinitrophenol, aminonitrophenol, hydroxyanisole, hydroxydinitrobenzene, dihydroxydinitrobenzene, alkylhydroxyanisole, hydroxynitroanisole, hydroxynitrobenzenecarboxylic acid (i.e., hydroxynitrobenzoic acid), dihydroxynitrobenzenecarboxylic acid (i.e., dihydroxynitrobenzoic acid), phenol, dihydroxybenzene, trihydroxybenzene, dihydroxybenzenecarboxylic acid, trihydroxybenzenecarboxylic acid, hydroxybenzenedicarboxylic acid, dihydroxybenzenedicarboxylic acid, hydroxytoluenecarboxylic acid, nitronaphthol, aminonaphthol, dinitronaphthol, hydroxynaphthalenecarboxylic acid, dihydroxy naphthalenecarboxylic acid, trihydroxynaphthalenecarboxylic acid, hydroxynaphthalene dicarboxylic acid, dihydroxynaphthalene dicarboxylic acid, hydroxyanthracene, dihydroxyanthracene, trihydroxyanthracene, tetrahydroxyanthracene, hydroxyanthracenecarboxylic acid, hydroxyanthracenedicarboxylic acid, dihydroxyanthracenedicarboxylic acid, tetrahydroxyanthracenedione, benzenecarboxylic acid, benzenedicarboxylic acid, naphthalenecarboxylic acid, naphthalene dicarboxylic acid, and the like.

Further, the high-boiling organic solvent having a boiling point of 150° C. or higher or the conductive auxiliary liquid can also include at least one binder selected from the group consisting of an epoxy compound or a hydrolyzate thereof, a silane compound or a hydrolyzate thereof, and a polyalcohol.

### EXAMPLES

Hereinafter, the present invention is described in more details based on the examples. However, it is noted that the following examples should not be used to narrowly construe the scope of the present invention.

### [Preparation Of Dopant Solution]

### Example 1

30 g of anthraquinone-2-sulfonic acid was dissolved in 70 g of water, followed by neutralizing it with 5 g of dimethylamine (pKb=11), thereby preparing a dopant solution containing a salt of anthraquinone-2-sulfonic acid and dimethylamine at a concentration of 5% by mass.

### Example 2

In the same manner as in Example 1, a dopant solution containing a salt of anthraquinone-2-sulfonic acid and dimethylamine at a concentration of 10% by mass was prepared.

### Example 3

In the same manner as in Example 1, a dopant solution containing a salt of anthraquinone-2-sulfonic acid and dimethylamine at a concentration of 30% by mass was prepared.

### Example 4

In the same manner as in Example 3 except that 8 g of diethylamine (pKb=11) was used instead of dimethylamine, a dopant solution containing a salt of anthraquinone-2-sulfonic acid and diethylamine at a concentration of 30% by mass was prepared.

### Example 5

In the same manner as in Example 3 except that 19 g of dihexylamine (pKb=11) was used instead of dimethylamine, a dopant solution containing a salt of anthraquinone-2-sulfonic acid and dihexylamine at a concentration of 30% by mass was prepared.

### Example 6

30 g of anthraquinone-1,5-disulfonic acid was dissolve in 70 g of water, followed by neutralizing it with 5 g of trimethylamine (pKb=10), thereby prepare a dopant solution containing 30% by mass of the salt of anthraquinone-1,5-disulfonic acid and trimethylamine.

### Example 7

In the same manner as in Example 6 except that 5 g of ethanolamine (pKb-9) was used instead of trimethylamine, a dopant solution containing a salt of anthraquinone-1,5-disulfonic acid and ethanolamine at a concentration of 30% by mass.

### Example 8

In the same manner as in Example 6 except that 7 g of diethylhydroxylamine (pKb-6) was used instead of trimethylamine, a dopant solution containing a salt of anthraquinone-1,5-disulfonic acid and diethylhydroxylamine at a concentration of 30% by mass was prepared.

### Example 9

In the same manner as in Example 3 except that 9 g of diethanolamine (pKb-9) was used instead of dimethylamine, a dopant solution containing a salt of anthraquinone-2-sulfonic acid and diethanolamine at a concentration of 30% by mass was prepared.

### Example 10

In the same manner as in Example 3 except that 16 g of triethanolamine (pKb=8) was used instead of dimethylamine, a dopant solution containing a salt of anthraquinone-2-sulfonic acid and triethanolamine at a concentration of 50% by mass was prepared.

### Example 11

In the same manner as in Example 3 except that 20 g of triisopropanolamine (pKb-9) was used instead of dimethylamine, a dopant solution containing a salt of anthraquinone-2-sulfonic acid and triisopropanolamine at a concentration of 60% by mass was prepared.

### Example 12

In the same manner as in Example 3 except that 9 g of 1-methylimidazole (pKb=7) was used instead of dimethylamine, a dopant solution containing a salt of anthraquinone-2-sulfonic acid and 1-methylimidazole at a concentration of 70% by mass was prepared.

### Example 13

In the same manner as in Example 11 except that the amount of water was changed, a dopant solution containing a salt of anthraquinone-2-sulfonic acid and triisopropanolamine at a concentration of 30% by mass was prepared.

### Comparative Example 1

A dopant solution was prepared by dissolving 1 g of sodium anthraquinone-2-sulfonate in 99 g of water, but much of the sodium anthraquinone-2-sulfonate remained undissolved, and thereby only obtain the dopant solution at a concentration of less than 1% by mass.

### Comparative Example 2

An attempt was made to prepare a dopant solution by dissolving 30 g of anthraquinone-2-sulfonic acid in 70 g of water, followed by neutralizing it with 2 g of methylamine, but it was found that the salt of anthraquinone-2-sulfonic acid and methylamine had a low solubility resulting in precipitation, and therefore a dopant solution could not be prepared.

### Comparative Example 3

An attempt was made to prepare a dopant solution by dissolving 30 g of anthraquinone-2-sulfonic acid in 70 g of water, followed by neutralizing it with 7 g of ammonia water at a concentration of 28% by mass, but it was found that the salt of anthraquinone-2-sulfonic acid and ammonia had a low solubility resulting in precipitation, and therefore, a dopant solution could not be prepared.

### Comparative Example 4

30 g of 2-naphthalenesulfonic acid was dissolved in 70 g of water, followed by neutralizing it with 11 g of butylamine (pKb=11), thereby preparing a dopant solution containing a salt of 2-naphthalenesulfonic acid and butylamine at a concentration of 30% by mass.

### Comparative Example 5

30 g of para-toluenesulfonic acid was dissolved in 70 g of water, followed by neutralizing it with 33 g of triisopropanolamine (pKb-9), thereby preparing a dopant solution containing a salt of para-toluenesulfonic acid and triisopropanolamine at a concentration of 30% by mass.

Table 1 shows the compositions of the dopant solutions of the Examples and the Comparative Examples. It is noted that Table 1 shows that the aromatic sulfonic acid (anthraquinone-2-sulfonic acid, etc.) and the neutralizing agent (dimethylamine, etc.) to constitute the dopant [the above salt (A), etc.] are listed separately (The same applies to Table 4 below). In addition, in the column of the aromatic sulfonic acids in Table 1, “AQS” stands for anthraquinone-2-sulfonic acid, “AQDS” stands for anthraquinone-1,5-sulfonic acid, “NS” stands for 2-naphthalenesulfonic acid, “PTS” stands for para-toluenesulfonic acid (the same applies to Table 4 below).

TABLE 1

| | dopant | | | |
|---|---|---|---|---|
| | aromatic | | | |
| | sulfonic | neutralizing agent | | Concentration |
| | acid | Kind | pKb | (mass %) |
| Example 1 | AQS | dimethylamine | 11 | 5 |
| Example 2 | AQS | dimethylamine | 11 | 10 |
| Example 3 | AQS | dimethylamine | 11 | 30 |
| Example 4 | AQS | diethylamine | 11 | 30 |
| Example 5 | AQS | dihexylamine | 11 | 30 |
| Example 6 | AQDS | trimethylamine | 10 | 30 |
| Example 7 | AQDS | ethanolamine | 9 | 30 |
| Example 8 | AQDS | diethylhydroxylamine | 6 | 30 |
| Example 9 | AQS | diethanolamine | 9 | 30 |
| Example 10 | AQS | triethanolamine | 8 | 50 |
| Example 11 | AQS | triisopropanolamine | 9 | 60 |
| Example 12 | AQS | 1-methylimidazole | 7 | 70 |
| Example 13 | AQS | triisopropanolamine | 9 | 30 |
| Comparative | AQS | (sodium) | — | <1 |
| Example 1 | | | | |
| Comparative | AQS | methylamine | 11 | 30 |
| Example 2 | | | | |
| Comparative | AQS | ammonia | 5 | 30 |
| Example 3 | | | | |
| Comparative | NS | butylamine | 11 | 30 |
| Example 4 | | | | |
| Comparative | PTS | triisopropanolamine | 9 | 30 |
| Example 5 | | | | |

### [Preparation Of Tantalum Electrolytic Capacitor]

### Example 14

A dielectric layer (dielectric oxide film) was formed on the surface of a tantalum sintered body by immersing the tantalum sintered body, which is a capacitor element, in 2% by mass of a phosphoric acid aqueous solution, followed by applying a voltage of 10V.

The tantalum sintered body above was immersed in the dopant solution prepared in Example 1, followed by taking it out and dried it at 105° C. for 10 minutes. The dried tantalum sintered body was immersed in an ethanol solution of EDOT having a concentration of 35% by mass, followed by taking it out after 1 minute, and leaving it for 5 minutes. Thereafter, this tantalum sintered body was immersed in an ammonium persulfate aqueous solution having a concentration of 30% by mass, followed by taking it out after 30 seconds and leaving it at room temperature for 30 minutes, and then it was heated at 50° C. for 10 minutes to perform polymerization. After polymerization, the tantalum sintered body above was immersed in water and left for 30 minutes, and then taken out and dried at 70° C. for 30 minutes. This operation was repeated six times to form a solid electrolyte layer made of the conductive composition on the surface of the capacitor element made of the tantalum sintered body.

Then, the solid electrolyte layer of the capacitor element was coated with carbon paste and silver paste, and then covered with an exterior material, thereby obtaining a tantalum electrolytic capacitor. It is noted that the design capacitance of the tantalum electrolytic capacitor of Example 1 was 250 μF (the same applies to the tantalum electrolytic capacitors and multilayer aluminum electrolytic capacitors of each of the Examples and the Comparative Examples described later).

### Examples 15 to 25 and Comparative Examples 6 and 7

Tantalum electrolytic capacitors were produced in the same manner as in Example 14, except that the dopant solutions were changed to those in Examples 2 to 12, or Comparative Examples 1 and 4.

The initial characteristics and the heat resistance of the tantalum electrolytic capacitors of Examples 14 to 25 and Comparative Examples 6 and 7 were evaluated using the following methods.

### (Initial Characteristics)

The capacitance of each tantalum electrolytic capacitor was measured at 120 Hz at 25° C. using an LCR meter (4284A) manufactured by HEWLETT PACKARD Corporation.

Also, the equivalent series resistance (ESR) of each tantalum electrolytic capacitor was measured at 100 kHz at 25° C. using an LCR meter (4284A) manufactured by HEWLETT PACKARD Corporation.

It is noted that the capacitance and the ESR described above were measured for 10 samples of each example, and the average value of the measured values of the 10 samples was rounded to the first decimal place.

### (Heat-Resistant)

Each of the 10 samples of the tantalum electrolytic capacitors of the Examples and the Comparative Examples was stored at 150° C. for 400 hours, followed by measuring the capacitance and the ESR in the same manner as described above, thereby obtaining the average value of each of the 10 measured values by rounding it to the first decimal place.

Also for the capacitance, the change rate (%) from the average value at the evaluation time of initial characteristic of the capacitance was obtained by the following formula. For the ESR, the rate of change (fold) was calculated by dividing the average value at the evaluation time of the heat resistance by the average value at the evaluation time of the initial characteristic.

Change rate (%) of the average value of the capacitance heat resistance evaluation from the average value of the initial characteristic evaluation:

$$Change rate ( \% ) = 100 \times ( heat resistance evaluation average value - initial characteristic evaluation average value ) \div Initial characteristic evaluation average value$$

The evaluation results above are shown in Table 2.

TABLE 2

| | | | | heat resistance | |
|---|---|---|---|---|---|
| | | initial characteristics | | change rate | change |
| | Dopant | capac- | | of capac- | rate of |
| | solution | itance | ESR | itance | ESR |
| | used | (μF) | (mΩ) | (%) | (fold) |
| Example 14 | Example 1 | 180.2 | 50.1 | −11.5 | 12.6 |
| Example 15 | Example 2 | 181.6 | 47.6 | −7.2 | 7.6 |
| Example 16 | Example 3 | 182.0 | 44.5 | −5.6 | 4.0 |
| Example 17 | Example 4 | 179.8 | 43.1 | −4.4 | 4.3 |
| Example 18 | Example 5 | 180.5 | 43.8 | −4.8 | 4.8 |
| Example 19 | Example 6 | 181.6 | 42.7 | −6.2 | 5.6 |
| Example 20 | Example 7 | 182.7 | 42.6 | −5.8 | 5.1 |
| Example 21 | Example 8 | 182.4 | 43.1 | −5.1 | 4.7 |
| Example 22 | Example 9 | 181.9 | 42.6 | −6.3 | 6.9 |
| Example 23 | Example 10 | 180.6 | 42.8 | −4.1 | 4.2 |
| Example 24 | Example 11 | 183.0 | 42.8 | −4.0 | 4.0 |
| Example 25 | Example 12 | 182.7 | 43.6 | −6.0 | 5.8 |
| Comparative | Comparative | 180.0 | 56.0 | −24.8 | 23.6 |
| Example 6 | Example 1 | | | | |
| Comparative | Comparative | 182.9 | 42.7 | −14.9 | 15.7 |
| Example 7 | Example 4 | | | | |

The tantalum electrolytic capacitor of Comparative Example 7 uses as a solid electrolyte a conductive composition formed by using a dopant solution containing conventionally known butylamine salt of naphthalene sulfonic acid as a dopant. The electrolytic capacitor of Comparative Example 7 is compared with the tantalum electrolytic capacitors of Examples 14 to 25 in which the solid electrolyte was a conductive composition formed using a dopant solution containing the above salt (A) as a dopant. While the capacitance and the ESR at the time of the initial characteristic evaluation were equivalent, it was found that the change rate of the capacitance and the ESR at the time of the heat resistance evaluation from the time of the initial characteristic evaluation was small, and therefore the Examples had excellent heat resistance.

Also, when comparing the electrolytic capacitors of Examples 14 to 16 in which only the concentration of the above salt (A) in the dopant solution was changed, the change rate of the capacitance and the ESR at the time of the heat resistance evaluation from the time of the initial characteristic evaluation was found that they were decreased in the order of Example 14, Example 15, and Example 16. Therefore, it was found that the higher the concentration of the above salt (A) in the dopant solution is, the higher the value of the heat resistance of the electrolytic capacitor obtained.

It is noted that the electrolytic capacitor of Comparative Example 6 included the solid electrolyte of a conductive composition formed using a dopant solution that contained sodium anthraquinone sulfonate as a dopant, and therefore its concentration could not be made high. Its change rate of the capacitance and the ESR at the time of the heat resistance evaluation from the time of the initial characteristic evaluation was found that they were bigger than not only the electrolytic capacitors of the Examples but also than the electrolytic capacitor of Comparative Example 7. Therefore, it was found that its heat resistance was inferior.

### Example 26

In the same manner as in Example 14 or the like, a tantalum sintered body with a dielectric layer (dielectric oxide film) formed on the surface thereof was immersed in the dopant solution prepared in Example 13 for 2 minutes, followed by pulling it out, and then it was dried at 105° ° C. for 10 minutes. The dried tantalum sintered body was immersed in an aqueous ferric sulfate solution having a concentration of 20% by mass, and dried at 105° C. for 10 minutes. The tantalum sintered body after drying was immersed in an ethanol solution of EDOT with a concentration of 35% by mass, followed by taking it out after 1 minute, and it was left to stand at room temperature for 30 minutes, and then it was heated at 50° C. for 10 minutes to perform polymerization. After polymerization, the tantalum sintered body above was immersed in water and left for 30 minutes, and then taken out and dried at 70° C. for 30 minutes. This operation was repeated six times to form a solid electrolyte layer made of the conductive composition on the surface of the capacitor element made of the tantalum sintered body. Then, the solid electrolyte layer of the capacitor element was coated with carbon paste and silver paste, and then covered with an exterior material, thereby obtaining a tantalum electrolytic capacitor.

### Example 27 and Comparative Examples 8

Tantalum electrolytic capacitors were produced in the same manner as in Example 26, except that the dopant solutions were changed to those in Example 7 or Comparative Example 5.

The tantalum electrolytic capacitors of Examples 26 and 27 and Comparative Example 8 were evaluated for the initial characteristics and the beat resistance in the same manner as the tantalum electrolytic capacitor of Example 14 or the like. The evaluation results above are shown in Table 3.

TABLE 3

| | | | | heat resistance | |
|---|---|---|---|---|---|
| | | initial characteristics | | change rate | change |
| | Dopant | capac- | | of capac- | rate of |
| | solution | itance | ESR | itance | ESR |
| | used | (μF) | (mΩ) | (%) | (fold) |
| Example 26 | Example 13 | 183.2 | 40.8 | −7.2 | 5.9 |
| Example 27 | Example 7 | 182.5 | 40.5 | −6.9 | 5.4 |
| Comparative | Comparative | 182.6 | 40.6 | −16.8 | 19.2 |
| Example 8 | Example 5 | | | | |

The tantalum electrolytic capacitors of Examples 26 and 27 and Comparative Example 8 used a conductive composition manufactured using ferric sulfate, which is an iron-based oxidizing agent, as a solid electrolyte. The tantalum electrolytic capacitors of Examples 26 and 27 using the dopant solution including the above salt (A) as a dopant, like the electrolytic capacitor of Example 14 or the like, had a change rate of the capacitance and the ESR at the heat resistance evaluation from the initial characteristic evaluation, which were smaller and found to be excellent in the heat resistance.

On the other hand, the electrolytic capacitor of Comparative Example 8 used a dopant solution containing a salt of paratoluenesulfonic acid which does not have an anthraquinone skeleton as a dopant, and the change rate of its capacitance and its ESR at the time of heat resistance evaluation were largely changed from the time of the initial characteristic evaluation, and therefore it was found that the heat resistance was poor.

### [Preparation Of Monomer Liquid]

### Example 28

25 g of EDOT, 30 g of a salt of anthraquinone-2-sulfonic acid and ethanolamine obtained by neutralizing anthraquinone-2-sulfonic acid with ethanolamine, and 45 g of methanol were mixed by stirring for 1 hour, thereby preparing a monomer liquid.

### Example 29

25 g of a mixture of EDOT and ethylated EDOT at 1:3 (mass ratio), and 30 g of a salt of anthraquinone-1,5-disulfonic acid and diethanolamine obtained by neutralizing anthraquinone-1,5-disulfonic acid with diethanolamine, and 45 g of ethanol were mixed with stirring for 1 hour, thereby preparing a monomer liquid.

### Example 30

25 g of a mixture of EDOT and propylated EDOT at 1:3 (mass ratio), 30 g of a salt of anthraquinone-1,5-disulfonic acid and triethanolamine obtained by neutralizing anthraquinone-1,5-disulfonic acid with triethanolamine, and 45 g of ethanol were stirred for 1 hour, thereby preparing a monomer liquid.

### Example 31

25 g of a mixture of EDOT and butylated EDOT at 1:3 (mass ratio), 30 g of a salt of anthraquinone-2-sulfonic acid and triisopropanolamine obtained by neutralizing anthraquinone-2-sulfonic acid with triisopropanolamine, and 45 g of butanol were mixed with stirring for 1 hour, thereby preparing a monomer liquid.

### Comparative Example 9

A monomer liquid was prepared in the same manner as in Example 28 except that anthraquinone-2-sulfonic acid was used instead of the salt of anthraquinone-2-sulfonic acid and ethanolamine.

### Comparative Example 10

An attempt was made by performing the same procedure as in Example 28 except for using sodium anthraquinone-2-sulfonate was used instead of the salt of anthraquinone-2-sulfonic acid and ethanolamine, and ethanol was used instead of methanol. However, a large amount of sodium anthraquinone-2-sulfonate remained undissolved, thereby making it impossible to prepare a monomer liquid containing it at a high concentration.

### Comparative Example 11

A monomer liquid was prepared in the same manner as in Example 28, except that a salt of 2-naphthalenesulfonic acid and butylamine was used instead of sodium anthraquinone-2-sulfonate, and ethanol was used instead of methanol.

Regarding the monomer liquids of Examples 28 to 31 and Comparative Examples 9 to 11, the composition regarding the dopant is shown in Table 4, and the composition regarding the monomer and the solvent is shown in Table 5. It is noted that in Table 5, “EDOT/Et-EDOT” means a mixture of EDOT and ethylated EDOT, “EDOT/Pr-EDOT” means a mixture of EDOT and propylated EDOT, and “EDOT/Bu-EDOT” means a mixture of EDOT and butylated EDOT, respectively.

TABLE 4

| | dopant | | | |
|---|---|---|---|---|
| | aromatic | neutralizing agent | | concentration |
| | sulfonic acid | Kind | pKb | (mass %) |
| Example 28 | AQS | ethanolamine | 9 | 30 |
| Example 29 | AQDS | diethanolamine | 9 | 30 |
| Example 30 | AQDS | triethanolamine | 8 | 30 |
| Example 31 | AQS | triisopropanolamine | 9 | 30 |
| Comparative | AQS | — | — | 30 |
| Example 9 | | | | |
| Comparative | AQS | (sodium) | — | 30 |
| Example 10 | | | | |
| Comparative | NS | butylamine | 11 | 30 |
| Example 11 | | | | |

TABLE 5

monomer

| | | concentration | |
|---|---|---|---|
| | kind | (mass %) | solvent |
| Example 28 | EDOT | 25 | methanol |
| Example 29 | EDOT/Et-EDOT | 25 | ethanol |
| Example 30 | EDOT/Pt-EDOT | 25 | ethanol |
| Example 31 | EDOT/Bu-EDOT | 25 | butanol |
| Comparative | EDOT | 25 | methanol |
| Example 9 | | | |
| Comparative | EDOT | 25 | ethanol |
| Example 10 | | | |
| Comparative | EDOT | 25 | ethanol |
| Example 11 | | | |

### [Preparation Of Multilayer Aluminum Electrolytic Capacitor]

### Example 32

An aluminum foil serving as a capacitor element was immersed in an ammonium adipate aqueous solution having a concentration of 2% by mass, and a voltage of 10V was applied to form a dielectric layer (dielectric oxide film) on the surface of the aluminum foil.

The aluminum foil above was immersed in the monomer liquid prepared in Example 28 for 2 minutes, followed by pulling it out, and then it was dried at 50° C. for 10 minutes. Next, the aluminum foil above was immersed in an ammonium persulfate aqueous solution having a concentration of 30% for 2 minutes, followed by taking it out after 30 seconds and leaving to stand at room temperature for 30 minutes, and then it was heated at 50° C. for 10 minutes to perform polymerization. After polymerization, the aluminum foil was immersed in water and left for 30 minutes, then taken out and dried at 70° C. for 30 minutes. This operation was repeated six times to form a solid electrolyte layer made of the conductive composition on the surface of the capacitor element made of aluminum foil.

Then, the solid electrolyte layer of the capacitor element was coated with carbon paste and silver paste, and then covered with an exterior material, thereby obtaining a multilayer aluminum electrolytic capacitor.

### Examples 33 to 35 and Comparative Examples 12 and 13

A multilayer aluminum electrolytic capacitor was produced in the same manner as in Example 32, except that the monomer liquid was changed to those of Examples 29 to 31 or Comparative Examples 9 and 11.

The multilayer aluminum electrolytic capacitors of Examples 32 to 35 and Comparative Examples 12 and 13 were evaluated for the initial characteristics and the heat resistance in the same manner as the tantalum electrolytic capacitor of Example 14 or the like. The evaluation results above are shown in Table 6.

TABLE 6

| | | | | heat resistance | |
|---|---|---|---|---|---|
| | | initial characteristics | | change rate | change |
| | Dopant | capac- | | of capac- | rate of |
| | solution | itance | ESR | itance | ESR |
| | used | (μF) | (mΩ) | (%) | (fold) |
| Example 32 | Example 28 | 181.4 | 42.7 | −4.5 | 4.6 |
| Example 33 | Example 29 | 182.4 | 43.1 | −5.4 | 5.6 |
| Example 34 | Example 30 | 180.9 | 42.4 | −5.1 | 4.9 |
| Example 35 | Example 31 | 180.2 | 42.8 | −4.2 | 4.5 |
| Comparative | Comparative | 182.8 | 42.2 | −30.4 | 32.9 |
| Example 12 | Example 9 | | | | |
| Comparative | Comparative | 183.5 | 41.4 | −13.2 | 14.0 |
| Example 13 | Example 11 | | | | |

The multilayer aluminum electrolytic capacitors of Examples 32 to 35 included a solid electrolyte of a conductive composition formed by using a monomer liquid containing the above salt (A) as a dopant. The electrolytic capacitor of Comparative Example 13 included a solid electrolyte of a conductive composition formed by using a conventionally known butylamine salt of naphthalene sulfonic acid. Compared to the formers with the latter, the capacitance and the ESR at the time of initial characteristic evaluation were equivalent, but the change rate of the capacitance and the ESR at the heat resistance evaluation from the time of initial characteristic evaluation was smaller. Therefore, it was found that the formers were excellent in the heat resistance.

It is noted that the electrolytic capacitor of Comparative Example 12 in which anthraquinone sulfonic acid was used as a dopant instead of the above salt (A) had a large change rate of the capacitance and the ESR at the heat resistance evaluation from the time of the initial characteristic evaluation, and therefore the heat resistance was found poor. This is considered to be because an acidity was increased to cause corrosion of the capacitor element when the conductive composition is formed on the surface of the capacitor element.

There can be provided other embodiments than the description above without departing the gist of the present invention. The embodiment described above is an example only, and the present invention is not limited to the specific embodiment. The scope of the present invention should be construed primarily based on the claims, not to the description of the specification or the present application. Any changes within the terms of the claims and the equivalence thereof should be construed as falling within the scope of the claims.

### INDUSTRIAL APPLICABILITY

The electrolytic capacitor of the present invention can be applied to the same applications as conventionally known electrolytic capacitors, but since it has excellent heat resistance, it can also be preferably applied to the applications where it is exposed to a high temperature. Also, the conductive composition of the present invention is suitable as a solid electrolyte for an electrolytic capacitor. Furthermore, the dopant solution for conductive polymers of the present invention and the monomer liquid for manufacturing conductive polymers of the present invention are suitable for manufacturing a conductive composition constituting a solid electrolyte of an electrolytic capacitor with excellent heat resistance.

1. A dopant solution for a conductive polymer, comprising a dopant for a conductive polymer, the dopant dissolved in a solvent,
wherein the dopant for the conductive polymer comprises a salt (A) of: a sulfonic acid having an anthraquinone skeleton; and one selected from the group consisting of an alkylamine represented by the following general formula (1), an alkanolamine represented by the following general formula (2), a hydroxylamine represented by the following general formula (3), and a compound having a heterocycle having 1 to 3 nitrogen atoms in a ring thereof,
wherein water or a lower alcohol is included as a solvent, 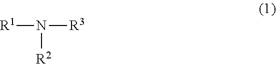
wherein in the above general formula (1), R¹ and R² are each an alkyl group having 1 to 6 carbon atoms, and R³ is a hydrogen atom or an alkyl group having 1 to 6 carbon atoms 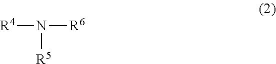
wherein in the above general formula (2), R⁴ is a hydroxyalkyl group having 1 to 6 carbon atoms, and each of R⁵ and R⁶ is a hydrogen atom, a hydroxyalkyl group having 1 to 6 carbon atoms, or an alkyl group having 1 to 6 carbon atoms 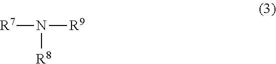
wherein in the above general formula (3), R⁷ is a hydroxyl group, and each of R⁸ and R⁹ is an alkyl group having 1 to 6 carbon atoms.

2. The dopant solution for the conductive polymer according to claim 1, wherein a concentration of the salt (A) is 5% by mass or more.

3. A monomer liquid for producing a conductive polymer comprising a monomer for producing the conductive polymer, and a dopant for the conductive polymer, wherein the dopant for the conductive polymer is dissolved,
wherein the dopant for the conductive polymer comprises a salt (A) of: a sulfonic acid having an anthraquinone skeleton; and one selected from the group consisting of an alkylamine represented by the following general formula (1), an alkanolamine represented by the following general formula (2), a hydroxylamine represented by the following general formula (3), and a compound having a heterocycle having 1 to 3 nitrogen atoms in a ring thereof, 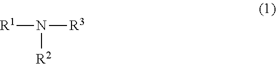
wherein in the above general formula (1), R¹ and R² are each an alkyl group having 1 to 6 carbon atoms, and R³ is a hydrogen atom or an alkyl group having 1 to 6 carbon atoms 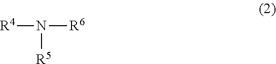
wherein in the above general formula (2), R⁴ is a hydroxyalkyl group having 1 to 6 carbon atoms, and each of R⁵ and R⁶ is a hydrogen atom, a hydroxyalkyl group having 1 to 6 carbon atoms, or an alkyl group having 1 to 6 carbon atoms 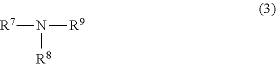
wherein in the above general formula (3), R⁷ is a hydroxyl group, and each of R⁸ and R⁹ is an alkyl group having 1 to 6 carbon atoms.

4. The monomer liquid for producing the conductive polymer according to claim 3, further comprising a lower alcohol as a solvent.

5. The monomer liquid for producing a conductive polymer according to claim 3, wherein monomer for producing the conductive polymer comprises at least one selected from the group consisting of thiophene or a derivative thereof, pyrrole or a derivative thereof, and aniline or a derivative thereof.

6. The monomer liquid for producing the conductive polymer according to claim 3, wherein a concentration of the salt (A) is 5% by mass or more.

7. A conductive composition obtained by an oxidation polymerization of the monomer for producing the conductive polymer in the presence of the dopant solution for the conductive polymer according to claim 1.

8. The conductive composition according to claim 7, wherein the monomer for producing the conductive polymer is at least one selected from the group consisting of thiophene or a derivative thereof, pyrrole or a derivative thereof, and aniline or a derivative thereof.

9. A conductive composition obtained by an oxidation polymerization of the monomer for producing the conductive polymer by using the monomer liquid for producing the conductive polymer according to claim 3.

10. A method for producing a conductive composition obtained by an oxidation polymerization of the monomer for producing the conductive polymer in the presence of the dopant solution for the conductive polymer according to claim 1.

11. A method for producing the conductive composition according to claim 10, wherein the monomer for producing the conductive polymer is at least one selected from the group consisting of thiophene or a derivative thereof, pyrrole or a derivative thereof, and aniline or a derivative thereof.

12. A method for producing a conductive composition obtained by an oxidation polymerization of the monomer for producing the conductive polymer by using the monomer liquid for producing the conductive polymer according to claim 3.

13. An electrolytic capacitor comprising a solid electrolyte,
wherein the conductive composition according to claim 7 is included as the solid electrolyte.

14. A method of producing an electrolytic capacitor comprising a solid electrolyte,
wherein the conductive composition produced by the method for producing the conductive composition according to claim 10 is used as the solid electrolyte.
